(19) United States
(12) Patent Application Publication
(10) Pub. No.: US 2007/0292372 A1
(43) Pub. Date: Dec. 20, 2007

(54) GEL COMPOSITION FOR IMPROVING ORAL HEALTH, SYSTEM INCLUDING THE COMPOSITION, AND METHODS OF USING AND FORMING SAME

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/749,144

(22) Filed: May 15, 2007

Related U.S. Application Data
(60) Provisional application No. 60/800,638, filed on May 15, 2006.

(51) Int. Cl. A61K 8/97; A61Q 11/00

(52) U.S. Cl. 424/58

(57) ABSTRACT

A composition for improving oral health, a system including the composition, a method of using the composition and system, and methods of forming the composition and system are disclosed. The composition is a gel that maintains contact with a portion of an oral cavity for an extended period of time to, for example, retard plaque and calculus buildup. The system includes a composition and a container encapsulating the composition. The composition is applied by rupturing the container and applying the composition through the ruptured portion of the container.

### CROSS REFERENCE TO RELATED APPLICATIONS

This application claims the benefit of U.S. Patent Application Ser. No. 60/800,638, entitled METHOD AND SYSTEM FOR FACILITATING ORAL HEALTH, filed May 15, 2006.

### FIELD OF INVENTION

The present invention generally relates to compositions and systems for improving oral health. More particularly, the invention relates to gel compositions for reducing an amount of and/or severity of plaque, calculus, bleeding, gingivitis, and other oral diseases and/or mitigating any increase of the same.

### BACKGROUND OF THE INVENTION

Unfortunately, poor oral health affects millions of people every year. Poor oral health may result in symptoms ranging from bad breath, tooth decay, and tooth coloration, to more serious health problems, such as gum disease, tooth loss, heart disease, stroke, poorly controlled diabetes and preterm labor.

The presence of dental plaque, or simply plaque, in an oral cavity can lead to such oral and general health problems. Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. Generally transparent and sticky, plaque attaches to hard surfaces such as teeth and dental devices. Once plaque forms on a surface, the plaque can usually be removed by brushing and flossing the affected areas. If not removed, however, the presence of plaque can give rise to tartar formation, tooth decay, gingivitis, periodontitis, and other health problems.

Tartar is a hard, calcified plaque material that exhibits a yellowish or brownish color. Tartar forms as a result of minerals (e.g., those present in saliva and gum pockets) reacting with plaque material to form a rough calculus. The calculus can form on surfaces not covered by the oral mucosa (supragingival) or on surface located apical to the soft tissue margin of the gingiva (subgingival).

Tartar adheres to hard surfaces such as enamel, roots, and dental devices, such as dentures, bridges, crowns, and the like, and is generally more difficult to remove than plaque. Brushing and flossing are normally not sufficient to remove tartar from a surface.

If left untreated, tartar buildup can be problematic in several regards. For example, the rough, porous surface of tartar serves as a breeding ground for additional bacteria, which can calcify and form additional tartar. The bacteria growth from both plaque and tartar can, in turn, lead to tooth decay, gum disease, gingivitis, periodontitis, and tooth loss, as well as systemic health problems.

Gingivitis is the beginning stage of periodontitis and is often caused by long-term effects of plaque and tartar buildup. Gingivitis is characterized by red, swollen gums. A periodontal probe will often measure about 3 to about 5 mm in depth into the space between the teeth and gums at the early stages of gingivitis. At this stage, gingivitis can often be reversed with proper treatment.

Left untreated, gingivitis will likely progress to advanced periodontitis. At this stage of gum disease, plaque and tartar are typically present supragingival and subgingival and an infection has destroyed bone around a tooth. Teeth often become loose, and the pocket depth may range between about 5 mm and about 8 mm at this stage.

As noted above, in addition to the health concerns, plaque and tartar may give rise to cosmetic problems. Tartar can be particularly problematic because it is difficult to remove and because of its yellowish or brownish color, which stains teeth and dental devices. Moreover, because the surface of tartar is rough and porous, tartar tends to absorb colors from other sources (e.g., coffee, tea, smoke, and the like), and thus the presence of tartar exacerbates cosmetic tooth coloration typically associated with such other sources.

Typical self-administered techniques for promoting oral health include brushing, flossing, and rinsing with a mouth rinse that typically includes an antimicrobial agent. Although such techniques may work relatively well at mitigating plaque buildup, such techniques are not thought to be effective at removing existing tartar from a surface.

The usual methods of preventing tartar buildup include brushing with a tartar-control toothpaste. Although such toothpastes, if used regularly, may prevent additional buildup of tartar, the toothpastes are not thought to be effective at removing existing tartar from tooth and device surfaces.

Typical methods of removing existing tartar typically include scaling or root planing, both of which are performed by dentists or hygienists with the aid of specialized tools. Although these techniques work well, they are relatively expensive and time consuming. Furthermore, various methods for removing tartar, such as those using pyrophosphate salts, often cause damage to tooth enamel and/or to dental devices.

Accordingly, improved compositions, methods, and systems for improving oral health are desired.

### SUMMARY OF THE INVENTION

The present invention provides a composition, system, and method for improving oral health. More particularly, the invention provides a gel composition configured to improve oral health, a system including the composition, and methods of using and forming the composition and system. As discussed in more detail below, the composition, system and method improve oral health by, for example, mitigating growth of plaque, tartar, and the like and/or reducing the presence of the same.

While the ways in which the invention addresses the various drawbacks of known compositions and methods will be described in more detail below, in general, the present invention provides a relatively inexpensive, safe, easy-to-use composition and system for the improvement of oral health. The system of the present invention and method of using the system do not require a visit to a dentist office and do not damage the surface of tooth enamel.

In accordance with various embodiments of the invention, the composition is a gel having a viscosity greater than about 20,000 centipoise (cp), preferably greater than 30,000 cp, and more preferably greater than about 35,000 cp. The viscosity of the compositions may range from about 20,000 cp to about 250,000 cp, preferably about 25,000 to about 100,000 cp, and more preferably about 30,000 cp to about 50,000 cp. The exemplary viscosity ranges facilitates maintaining the composition in contact with an oral surface for an extended period of time, while still allowing application of the composition using systems described herein. In accordance with various aspects of these embodiments, the composition includes a humectant. In accordance with further aspects, the composition includes an ingredient to facilitate migration of materials through the composition.

In accordance with further embodiments of the invention, the composition includes a carrier, a thickener, and one or more antimicrobial agents. In accordance with various aspects of these embodiments, the composition may include a humectant, migration enhancing agents, oils, and the like.

In accordance with yet further embodiments, the composition includes a preservative. In accordance with various aspects of these embodiments, the composition may include a humectant, migration enhancing agents, oils, and the like.

In accordance with yet additional embodiments, a composition includes coloring indicative of healthy gingiva.

In accordance with further embodiments of the invention, a system includes a composition and a container for enclosing and applying the composition.

In accordance with embodiments of the invention, a system includes a viscous composition and a container configured to dispense the viscous composition. In accordance with various aspects of these embodiments, the viscous composition is designed to maintain contact with a surface for an extended period of time. Exemplary compositions have a viscosity greater than about 20,000 cp, preferably greater than 30,000 cp, and more preferably greater than about 35,000 cp. The viscosity of the compositions may range from about 20,000 cp to about 250,000 cp, preferably about 25,000 cp to about 100,000 cp, and more preferably about 30,000 cp to about 50,000 cp. In accordance with additional aspects of these embodiments, the composition includes a humectant, migration enhancing agents, oils, and the like. In accordance with additional aspects of this embodiment, the container encapsulates multiple doses of the composition. In accordance with alternative aspects, the system includes a single-dose applicator. In accordance with yet a further aspect, the container is configured to facilitate application of the composition to specific areas within an oral cavity. In accordance with yet additional aspects of this embodiment, the system is designed to improve oral health by, for example, reducing an index such as GI, PI, VM-I, SI, GBI, x-ray analysis, and probe pocket depth.

In accordance with additional embodiments of the invention, a method of improving oral health includes applying a composition to a portion of an oral cavity. In accordance with various aspects of these embodiments, the composition is applied on or near a tooth-gum interface. In accordance with further aspects, the composition is applied during or prior to a reduced saliva production time-for example, prior to extended periods of rest or sleep.

In accordance with an additional embodiment of the invention, a method of improving oral health includes using a system of the present invention. In accordance with various examples of this embodiment, the composition is dispensed from a sealed container by rupturing the sealed container and applying a single dose of the composition directly to the surface. In accordance with further aspects, the container includes multiple doses (e.g., 2, 4, or 7) doses of the composition. In accordance with another example, the composition is applied after brushing, flossing, and/or rinsing, and is applied before bedtime to allow the active ingredients to stay in contact with a surface for an extended period of time. In accordance with further aspects, a portion of the composition is expelled from an oral cavity prior to the extended period of rest or sleep.

In accordance with yet additional embodiments of the invention, a method of forming a composition for improving oral health includes the steps of adding a humectant to a vessel, adding a thickener to the vessel, mixing until a uniform, lump-free mixture is formed, adding a diluent to the mixture, adding sweeteners, flavorings, and colorants to the mixture, and adding oils to the mixture.

In accordance with yet further embodiments of the invention, a kit includes a plurality systems and method includes use of the kit.

### BRIEF DESCRIPTION OF THE DRAWINGS

The exemplary embodiments of the present invention will be described in connection with the appended drawing figures in which like numerals denote like elements and:

FIG. 1 illustrates a perspective view of a dispensing device suitable for use in accordance with exemplary embodiments of the invention;

FIGS. 2a-2c illustrate, respectively, a left view, a rear view, and a right view of the dispensing device illustrated in FIG. 1; and

FIGS. 3 and 4 illustrate other exemplary dispensing devices for use with the present invention.

Elements in the figures are illustrated for simplicity and clarity and have not necessarily been drawn to scale. The dimensions of some of the elements in the figures may be exaggerated relative to other elements to help to improve understanding of embodiments of the present invention.

### DETAILED DESCRIPTION

The present invention provides an oral care composition and system to improve oral health. More particularly, the invention provides a gel composition and system including the composition to improve oral health, such as improving (lowering) one or more oral health care indices such as GI, PI, VM-I, SI, GBI, x-ray analysis, and probe pocket depth. The system and method of the invention can be used to improve oral health of various animals, and are particularly well suited for the treatment of humans.

As used throughout this application, the term “surface” includes any surface on which plaque, tartar, or gum disease may form. Exemplary surfaces include teeth (both supragingival and subgingival), gums, and dental devices such as bridges, crowns, fillings, braces, and the like. Further, as used herein, the term “measurably improve” means a measurable difference between an amount measured without use of the composition or system of the present invention and with or after use of the system. The measurements may be compared for the same surface (before and after) or between test and control groups.

Viscous Composition

In accordance with various embodiments of the invention, a composition for improving oral health is a viscous gel configured such that a portion of the composition remains in contact with a surface within an oral cavity for an extended period of time. As noted above, providing a viscous composition in accordance with various embodiments of the invention retards formation of plaque, tartar, disease and/or disrupts and/or facilitates disruption of existing plaque, tartar, and/or the like.

In accordance with an exemplary embodiment, the composition also includes a thickener to obtain the desired viscosity. Suitable thickening agents include substances which increase the viscosity of the composition, cause the composition to gel or coagulate, or the like, such as food-grade or pharmaceutical-grade thickeners, including, for example, hydroxyethylcellulose, hydroxypropyl methylcellulose, carrageenan, guar gum, methylcellulose, methyethylecellulose, acceptable non-ionic thickeners, and the like. The thickener may be present in an amount of about 0.01% to about 10%, in an amount of about 0.1% to about 7%, or even in an amount of about 1% to about 5% or about 0.5% to about 3%.

The composition may also include a humectant such a glycerin, which may be present in an amount of about 0.01% to about 15%, preferably about 0.1% to about 10%, and more preferably about 1 to about 7%. When used, the humectant may facilitate maintaining the composition in a liquid form and may help maintain a desired viscosity of the composition. In addition, glycerin may facilitate maintaining one or more of the ingredients in an ionic form and may facilitate the transport of the ingredients and/or particulates through the composition.

The composition may also include a diluent. Exemplary diluents suitable for use with the present composition include sorbitol, xylitol, mannitol, water, alcohols, and oils. In accordance with particular examples of the invention, the composition includes purified water in an amount of about 80% to about 99%, preferably about 85% to about 95%, and more preferably about 88% to about 92%.

The composition may also include sugar alcohols such as sorbitol and xylitol, mormital, lactitol, and the like that act as a sweetener and also as a humectant and/or emulsifier and/or diluent. When used, sorbitol or other sugar alcohol can be present in an amount of about 0.001% to about 0.5%, in an amount of about 0.01% to about 0. 1%, or even in an amount of about 0.025% to about 0.075%. Compositions in accordance with the invention may alternatively include a greater percentage of sugar alcohol(s).

The composition may also include a natural or artificial sweetener such as cyclamates, sucralose, saccharin (e.g., sodium or calcium), ace-k, or aspartame which, when included in the composition, can be present in an amount of about 0.001% to about 1.5%, in an amount of about 0.01% to about 1%, or even in an amount of about 0.25% to about 0.75%.

Colorants may also be added to the composition. For example, the composition can include colorants, such that when the composition is applied to or proximate the gingiva, the composition has a color indicative of healthy gingiva—e.g., the composition can be pink in color. Such a composition having a color indicative of healthy gingiva can provide added incentive to users to continue using the composition, which in turn promotes improved health care and hygiene. Colorants may be present in any desired amount. For example, the colorants may include Red #33 and/or Red #40, available from Pylam in an amount of about 0.000005% to about 1%, preferably about 0.00050% to about 0.5%, and more preferably about 0.001% to about 0.1%. Additionally or alternatively, colorants, which are indicative of flavor may be added to the composition. Examples include FD&C Blue #1, D&C Green #5, FD&C Yellow #5, and FD&C Yellow #6.

The composition also optionally includes oils, such as essential oils—e.g., cinnamon bark oil, clove bud oil, mint flavorings, citrus flavorings, and the like. Clove bud oil includes ugenol, eugenyl acetate, caryophyllene, salicylic acid, and has local anesthetic, antiseptic, antibacterial, and stimulating properties. Cinnamon bark oil exhibits antibacterial, antiseptic, antiviral, antispasmodic, antifungal, sedative and analgesic properties. By way of one particular example, a composition includes cinnamon bark oil present in an about of about 0.001% to about 2%, preferably about 0.01% to about 1%, and more preferably about 0.1% to about 0.5%; and clove bud oil in an amount of about 0.001% to about 2%, preferably about 0.002% to about 1%, and more preferably about 0.003% to about 0.0075%.

A composition in accordance with various embodiments of the invention may also include a preservative or antimicrobial agent. Exemplary preservative and antimicrobial agents suitable for use in compositions of the present invention include methylparaben, and the like. Exemplary compositions include a preservative in an amount of about 0.001% to about 1%, preferably about 0.01% to about 0.5%, and more preferably about 0.05% to about 0.2%.

As noted above, the composition is configured to maintain contact with a surface for an extended period of time, which has several advantages over traditional compositions used to promote oral health. For example, because the composition is viscous and “sticks” to a surface to which it is applied, relatively small amounts of the composition can be used to effectively improve oral health—e.g., by preventing or removing plaque and/or tartar buildup on a surface. This is advantageous, not only from an economic viewpoint, but it also reduces an amount of the composition that may be ingested. Additionally, the composition of the composition enables easy delivery of the composition from its container, while also facilitating maintaining the composition in contact with particular areas within an oral cavity, such as the crevices within teeth and between the teeth and gingiva. In accordance with further embodiments of the invention, the viscosity is low enough to allow application of the composition to desired locations within the oral cavity.

Exemplary compositions have a viscosity greater than about 20,000 cp, preferably greater than 30,000 cp, and more preferably greater than about 35,000 cp. By way of more particular examples, the viscosity of compositions range from about 20,000 cp to about 250,000 cp, preferably about 25,000 cp to about 100,000 cp, and more preferably about 30,000 cp to about 50,000 cp, and yet more preferably about 35,000 cp to about 45,000 cp. The viscosity values as set forth herein are measured using a Brookfield, Model DV-II+ Pro viscometer, spindle # 6, 10 RPM for 90 seconds at 25 C.

A pH of composition 102 may vary in accordance with a particular application. In accordance with various embodiments of the invention, the pH is between about 4-10, preferably about 4-7, and more preferably about 5-5.4.

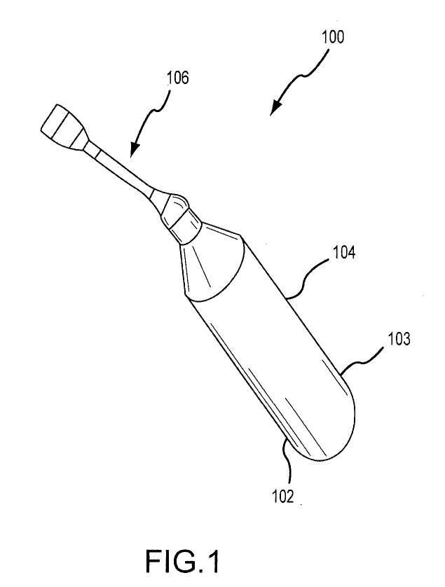

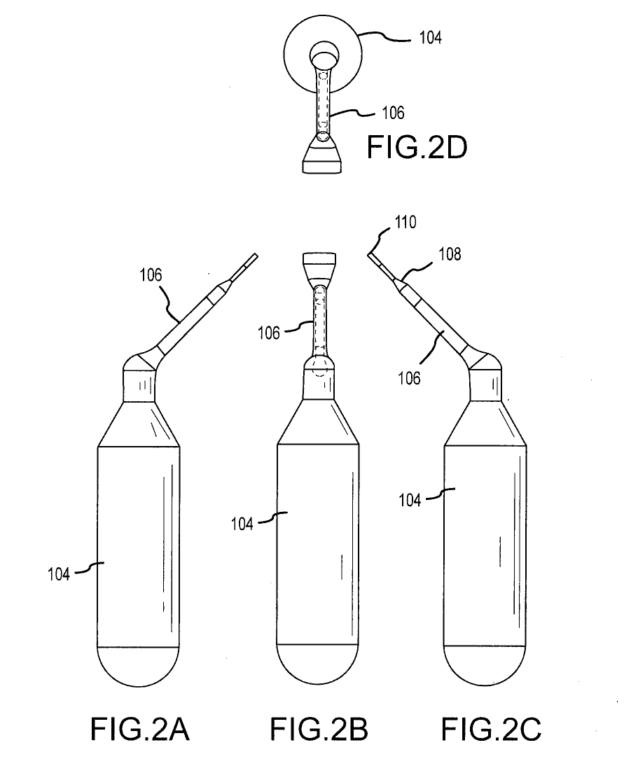

FIG. 1 and FIGS. 2a-2c illustrate a dispensing system 100 suitable for applying a composition 102 to a surface. System 100 includes a resilient containment portion 104 and an applicator portion 106. Applicator portion 106 further includes a severed or scarred area 108 and a sealed end 110.

Composition 102 is dispensed from system 100 by severing or rupturing a portion of device 100 (e.g., at or near scarred area 108) to expose composition 102 contained within system 100. Composition 102 is then expelled through applicator 106 to a desired area—e.g., a tooth gum interface of a user—by squeezing resilient portion 104 to cause the composition to flow from resilient portion 104 through applicator 106.

As noted above, system 100 may be configured to apply a composition to a specific area within an oral cavity. In this case, applicator 106 may have a desirably small cross-section across applicator 106. In accordance with specific examples of the present invention, a cross-sectional dimension, taken along line A-A, is about 0.001″ to about 0.2″, preferably about 0.05″ to about 0.01″, and more preferably about 0.06″ to about 0.09″.

Resilient portion 103 is designed to fit comfortably between two fingers (e.g., a thumb and an index finger) of a user, such that the composition is expelled by pressing the two fingers, on opposite sides of portion 104, together. In accordance with various examples of the invention, portion 104 has a cross-sectional dimension, taken along line B-B, of about 0.1″ to about 2″, preferably about 0.25″ to about 1″, and more preferably about 0.4″ to about 0.6″.

A single-dose size may vary in accordance with several factors, such as the particular ingredients, the dilution of the composition, and the like. Exemplary dose sizes for purpose of illustration range from about 1 mg to about 6 mg, preferably about 2 mg to about 5 mg, and more preferably about 3 mg to about 4 mg.

Containers described herein may be formed of any suitable material. Exemplary resilient materials include low density polyethylene material, high density polyethylene, medium density polyethylene, linear low density polyethylene, polyvinyl chloride, K resin, polyethylene terephthalate and copolyesters, polypropylene, surlyn, silicones and other thermostatics. The material may be opaque, transparent, or semitransparent. An advantage of forming containers of transparent or semitransparent material is that an amount of material (e.g., material 102) within a vessel portion can be ascertained when the portion is formed of such material. Material used to form the containers may also include UV protection additives, colorants, or the like, and is preferably FDA-approved material.

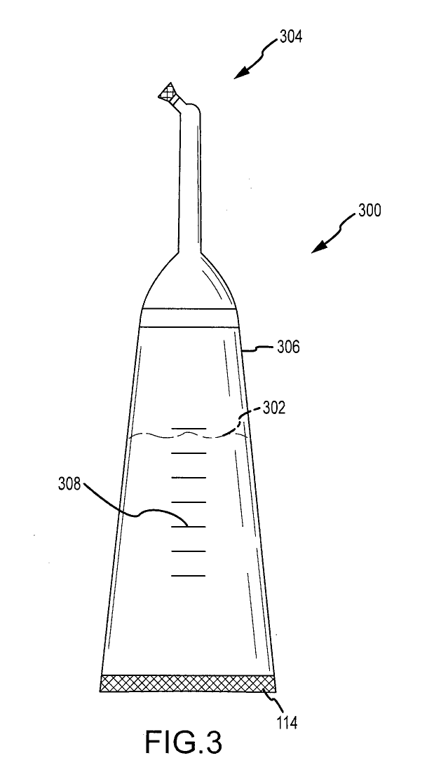

FIG. 3 illustrates a system 300 in accordance with further embodiments of the invention. System 300 is similar to system 100, except system 300 includes a separate top portion 304, which (optionally disengageably) couples to a resilient container portion 306. Both system 100 and system 300 may be configured as multi-dose or single-dose containers. When configured as a multi-dose container, as illustrated, a system may include graduated measurement marks 308 to indicate a dose size and to indicate a number of doses already used and/or a number of doses remaining. Containers in accordance with additional examples of the invention may include any suitable number of doses, such as 1, 2, 4, 7, or the like.

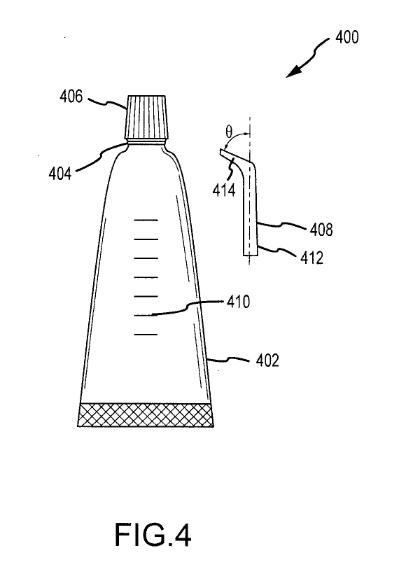

FIG. 4 illustrates another system 400 for use in accordance with additional embodiments of the invention. As illustrated, system 400 includes a vessel portion 402, including a neck 404, a cap 406, and a detachable applicator 408. System 400 is similar to system 300, except system 400 includes resealable cap 406 and detachable applicator 408, rather than applicator portion 304. System 400 may be formed of any of the materials noted above in connection with system 100, and may include graduations 410 to indicate a number of doses used and/or a number of remaining doses, as described above.

Cap 406 can be removably attached to vessel portion 402 using a variety of techniques. For example, cap 406 may be threadedly attached to portion 402. Alternatively, cap 406 and portion 402 may be coupled using snap-fit, lug, interference-fit technology, or similar technologies. In accordance with one specific example of this embodiment, neck 404 includes exterior threads and cap 406 includes interior threads to threadedly engage with neck 404.

Similarly, applicator portion 408 may couple to vessel 402 in a variety of ways, such at threaded, snap-fit, lug, or similar type connections. By way of particular example, applicator 408 threadedly engages with an interior portion of neck 404.

Applicator 408 includes a first portion 412 and a second or tip portion 414. As illustrated, tip portion 414 is angled relative to a centerline first portion 412; however such is not required for practice of the present invention. Exemplary angles range from about zero to about ninety degrees relative to the centerline.

Although not illustrated, systems in accordance with various embodiments of the invention may include tamper-resistant features. For example, system 400 may include a seal formed over neck 404, using, for example plastic or foil glued to or otherwise adhered to a top portion of neck 404.

Kits in accordance with various embodiments of the invention include one or more systems—e.g., systems 100, 300, 400, or the like. By way of one particular example, a kit includes four seven-dose systems.

Method of Improving Oral Health

A method of improving oral health includes applying a composition described herein to a surface and maintaining the composition in contact with the surface for an extended period of time.

The composition may be applied using a variety of methods such as with a swab, with a syringe, or using any other means capable of dispensing the composition. In accordance with various aspects of this embodiment, the composition is selectively applied using system 100, 300, or 400. By way of particular example, the composition 102 may be applied though chute 106 to, e.g., a tooth/gingival interface, such that the composition contacts both supragingival and subgingival areas where plaque and tartar are likely to form. Unlike most methods of promoting oral health, the methods of various aspects of this embodiment allow for direct, selective application of the composition to desired areas of a surface. Thus, less composition may be required to perform the task of improving oral health, because the composition is not ubiquitously applied to an oral cavity.

In accordance with further aspects, the composition is applied prior to or during a period of reduced saliva production. For example, the composition may be applied prior to extended (e.g., greater than 2 hours) periods of rest or before sleep. After application, a portion of the applied composition is expelled from the oral cavity, such that only a portion of the initially-applied composition remains in the oral cavity prior to the rest/sleep.

As set forth in more detail below, in accordance with one aspect of this embodiment, a measurable difference of a gingivitis index (e.g., the Loe and Silness Gingival Index (1963)) is obtained by using the composition and/or system of the present invention. More particularly, a measurable difference in the GI is obtained by applying a composition to an area (e.g., proximate an inflamed gum tissue, such as at the gum/tooth interface near the inflamed area). In accordance with various aspects of this embodiment, a single dose of the composition is applied after brushing teeth, flossing between teeth, and/or rinsing an oral cavity, and is applied such that the composition remains in contact with the affected area for an extended period of time. By way of particular example, the composition can be applied at bedtime to mitigate disruption of the composition/surface interface and to facilitate maintaining the composition in contact with the surface for an extended period.

In accordance with another aspect, a measurable difference of a plaque index, e.g., the Turesky Modification (1970) of the Quiqley and Hein Plaque Index (PI) is obtained by using the composition and/or system of the present invention. The composition is applied as described above in connection with obtaining a measurable difference in the GI.

In accordance with yet a further aspect of this exemplary embodiment, a measurable difference in a tartar index—e.g., the Volpe-Manhold Index (V-MI)—is obtained by using the composition and/or system of the present invention as described herein.

In accordance with another embodiment of the invention, an amount of tarter present on a surface is reduced using the composition and/or system of the present invention.

In accordance with yet another embodiment, a measurable improvement in a stain index—e.g., the Lobene Stain Index (SI)—is obtained using the composition and/or system of the present invention.

In accordance with yet a further embodiment, a measurable improvement in a gingival bleeding index (GBI) is obtained using the composition and/or system.

In accordance with yet another embodiment, a measurable improvement in oral health, as measured using intra-oral photography analysis, is obtained using the composition and/or system.

In accordance with yet a further embodiment, a measurable improvement in probe pocket depth is obtained using the composition and/or system as described herein.

In accordance with yet further embodiments, maintenance of periodontal health is maintained through use of the composition and/or system of the present invention.

Method of Forming a Composition

A method of forming a composition in accordance with various embodiments of the invention includes the steps of adding a humectant (e.g., glycerin) to a first mixing vessel and the adding a thickener (e.g., hydroxyethylcellulose) to the humectant and mixing until a uniform, lump-free slurry forms. The slurry should not sit for too long at this stage, or it may become cement-like in texture and viscosity. In a second mixing vessel, add a diluent (e.g., water) and add the humectant/thickener slurry slowly (over a period of a few hours) to the diluent and mix until a smooth mixture is obtained. Once the gum is hydrated, add any sugar alcohol, sweetener, and colorant to the mixture and mix until each is dissolved. Finally, add any oils and mix until the oils are dispersed in the solution.

### SPECIFIC EXAMPLES

The following non-limiting examples illustrate use of the system and method in accordance with various embodiments of the invention to improve oral health. These examples are merely illustrative, and it is not intended that the invention be limited to the examples. Systems in accordance with the present invention may include the ingredients listed below as well as additional and/or alternative inert materials, preservatives, and other constituents typically found in compositions for promoting oral health. In the case where exemplary inert materials and/or preservatives are listed, these ingredients are merely exemplary, and it is understood that other similar ingredients may be substituted for the materials listed in the examples below.

### Example 1

A pale light pink viscous gel, having a viscosity of about 40,000 cp, with cinnamon-clove characteristic odor and taste is formed by admixing the following ingredients as described above, in the amounts shown.

TABLE 1

| | | | Exemplary |
|---|---|---|---|
| Ingredient | Supplier | Weight % | Wt % Range |
| Purified Water | Copacker | 92.195 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Methylparaben | | 0.100 | 0.001-1 |
| Cinnamon Bark | Berje | 0.250 | 0.001-2 |
| (Oil) | | | |
| Clove Bud (Oil) | Berje | 0.005 | 0.001-2 |
| Red #40 | Pylam | 0.099 | 0-1 |
| (1% Solution) | | | |

A clinical study comparing V-MI scores for 52 people using the composition of Table 1 was conducted. During the three-month study, the subjects were instructed to brush with toothpaste twice daily and apply the composition prior to retiring.

The subjects received professional prophylaxis, including scaling and polishing, to remove supragingival calculus, extrinsic stain, and plaque deposits from the mandibular anterior teeth and initial V-MI measurements at 18 VM sites per subject were recorded prior to the study. Subjects were requested to refrain from flossing the mandibular 6 anterior teeth and place the composition between and around those teeth using system 100, illustrated in FIG. 1. At the end of a three-month period, VM-I measurements for the same teeth were recorded and analyzed using analysis of covariance. The V-MI scores at the end of the three-month period were statistically significantly lower than the initial V-MI scores (41% lower, p value=4.64E-18).

### Example 2

A clinical study comparing Gingivitis Index (GI) scores using the composition of Table 1 was conducted. 45 subjects were evaluated over a period of three months and 38 people were evaluated over a period of six months. During the study, the subjects were instructed to brush with toothpaste twice daily and apply the composition prior to retiring. The subjects were asked not to use other oral hygiene products. After a baseline examination, all subjects received dental prophylaxis.

The method of Loe-Silness (1963) was used to evaluate gingivitis. The method was conducted by the same examiner at each evaluation period according to the following criteria:

0=absence of inflammation

1=mild inflammation: slight change in color and texture

2=moderate inflammation: moderate redness, edema, glazing, hypertrophy; bleeding on probing

3=severe inflammation: marked redness and hypertrophy, a tendency to spontaneously bleeding (elicited by air syringe) and/or ulceration

At the end of a three-month period, a 18% reduction is the GI was observed and at the end of the six-month period, a 26% reduction in GI was observed.

### Example 3

In another study including 25 subjects, the subjects were screened and baseline measurements made. At the end of a six-week trial, a measurable improvement in GI mean score (19.1%), as measured using the Loe-Silness (1963) method described above, was observed.

### Example 4

In another study including 25 subjects, the subjects were screened and baseline measurements made. At the end of a six-week trial, an improvement in PI (33%) (Quigley Hein modified Turesky et al.) mean score was observed.

### Example 5

A clinical study, comparing Plaque Index (Quigley Hein modified Turesky et al.) (PI) scores using the composition of Table 1, was conducted. Forty-five subjects were evaluated over a period of three months and 38 people were evaluated over a period of six months. During the study, the subjects were instructed to brush with toothpaste twice daily and apply the composition prior to retiring. The subjects were asked not to use other oral hygiene products. After a baseline examination, all subjects received dental prophylaxis.

The method was conducted by the same examiner at each evaluation period according to the following criteria:

0=no plaque present

1=separate flecks of plaque at the cervical margin of the tooth

2=a thin, continuous band of plaque (up to a millimeter) at the cervical margin

3=a band of plaque wider than one millimeter but covering less than one-third of the surface

4=plaque covering at least one-third but less than two-thirds of the surface

5=plaque covering two-thirds or more of the surface

At the end of a three-month period, a 23% reduction is the PI was observed and at the end of the six-month period, a 26% reduction in PI was observed.

### Example 6

A clinical study comparing Bleeding Index (Eastman Bleeding Index) (GBI) scores using the composition of Table 1 was conducted. Forty-five subjects were evaluated over a period of three months and 38 people were evaluated over a period of six months. During the study, the subjects were instructed to brush with toothpaste twice daily and apply the composition prior to retiring. The subjects were asked not to use other oral hygiene products. After a baseline examination, all subjects received dental prophylaxis.

Gingival bleeding on probing was examined using the Eastman Bleeding Index (Caton and Polson, 1985) according to the following criteria.

0=no bleeding

1=presence of bleeding

At the end of a three-month period, a 67% reduction is the GBI was observed and at the end of the six-month period, a 65% reduction in GBI was observed.

### Example 7

In a study including 25 subjects, the subjects were screened and baseline measurements made. At the end of a three-week period and at the end of a six-week period there was a general reduction in plaque thickness and quality noticed in some of the subjects. This finding is significant because the presence of actively growing plaque bacteria (bioflim) may lead to the development of inflammation, which leads to gingivitis and periodontitis.

### Example 8

V-MI measurements for all mandibular incisors and cuspids were recorded at a baseline screening for 29 subjects. Subjects were provided toothbrushes and toothpaste and a system including a composition in accordance with the present invention. The subjects were instructed to apply the gel daily—once before going to bed. After applying the gel, the subjects were asked to spit out any excess and not eat or drink before going to bed. Subjects were also asked to not floss their lower 6 anterior teeth for the duration of the study, except to remove impacted food. Subjects were instructed to brush twice per day. At the end of a forty-day period, an 8% mean reduction in calculus was observed. At the end of a ninety-day period a 23% reduction of calculus was observed. The effectiveness of the composition appeared to increase in patients with more baseline calculus.

Subjects also believed that their gums were pinker, felt less tartar, and showed a decrease in tartar as analyzed using intraoral images, as a result of using the composition as set forth in Table 1 for about 90 days.

Although exemplary embodiments of the present invention are set forth herein, it should be appreciated that the invention is not so limited. Various modifications, variations, and enhancements in composition and method set forth herein may be made without departing from the spirit and scope of the present invention.

I claim:

1. A composition to improve oral health care, the composition comprising:
about 80 wt % to about 99 wt % diluent;
about 0.01 wt % to about 15 wt % humectant;
about 0.01 wt % to about 10 wt % thickener; and
about 0.001 wt % to about 2 wt % of a first oil.

2. The composition of claim 1, wherein the diluent comprises purified water.

3. The composition of claim 1, wherein the humectant comprises glycerin.

4. The composition of claim 1, wherein the thickener comprises a material selected from the group consisting of hydroxyethylcellulose, glycerin, carrageenan, guar gum, methylcellulose, and methyethylecellulose.

5. The composition of claim 1, wherein the first oil is an essential oil.

6. The composition of claim 1, wherein the first oil is selected from the group consisting of cinnamon bark oil and clove bud oil.

7. The composition of claim 1, further comprising a second oil.

8. The composition of claim 7, wherein the second oil is an essential oil.

9. The composition of claim 7, wherein the second oil is selected from the group consisting of cinnamon bud oil and clove bud oil.

10. The composition of claim 1, further comprising about 0.001 wt % to about 0.5 wt % sugar alcohol.

11. The composition of claim 1, further comprising about 0.001 wt % to about 1 wt % preservative.

12. The composition of claim 1 1, wherein the preservative is methylparaben.

13. A system to improve oral health, the system comprising:
a gel composition including a diluent, a thickener, and a plurality of oils, the composition having a viscosity greater than about 20,000 cp; and
a container comprising a resilient portion and an applicator portion, wherein the container is configured to directly apply the composition to a surface within an oral cavity.

14. The system of claim 13, wherein the viscosity of the composition is about 25,000 to about 100,000.

15. The system of claim 13, wherein the viscosity of the composition is about 30,000 to about 50,000 cp.

16. The system of claim 13, wherein the plurality of oils comprises an essential oil.

17. The system of claim 16, wherein the plurality of oils includes cinnamon bark oil.

18. The system of claim 16, wherein the plurality of oils includes clove bud oil.

19. The system of claim 13, wherein the gel composition further comprises a preservative.

20. A method of improving oral health, the method comprising the steps of:
providing a system including a composition having a viscosity of greater than about 20,000 cp and a container encasing the composition;
rupturing a portion of the container to form a ruptured portion; and
applying the composition through the ruptured portion to a portion of an oral cavity.
